(19) United States
(12) Patent Application Publication
(10) Pub. No.: US 2007/0129564 A1
(43) Pub. Date: Jun. 7, 2007

(54) Method for the production of (meth)acrylic acid esters

(75) Inventors: Reinhold Schwalm, Wachenheim (DE); Barbel Meyer, Lauta (DE); Helmut Gruner, Schwarzheide (DE); Thomas Daniel, Waldsee (DE); Ulrich Riegel, Landstuhl (DE); Dieter Hermeling, Bohl-Iggelheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/588,488

(22) PCT Filed: Feb. 16, 2005
(86) PCT No.: PCT/EP05/01533

§ 371 Date: Aug. 4, 2006

(30) Foreign Application Priority Data
Feb. 20, 2004 (DE) 10 2004 018 379.1

(51) Int. Cl. C07C 69/00

(52) U.S. Cl. 560/4

(57) ABSTRACT

Process for preparing (meth)acrylic esters, in which chromanol derivatives are used as stabilizers against polymerization, and the use of the thus obtainable (meth)acrylic esters.

The present invention relates to a process for preparing (meth)acrylic esters, in which chromanol derivatives are used as stabilizers against polymerization, and the use of the thus obtainable (meth)acrylic esters.

It is known that (meth)acrylic acid and (meth)acrylic esters can readily be polymerized by heat or the action of light or free-radical formers. However, since polymerization has to be reduced or prevented in the course of preparation, workup and/or storage for safety and economic reasons, there is a constant need for novel, effective polymerization inhibitors.

Especially for the preparation of polyfunctional (meth)acrylic esters which are used industrially, for example, as free-radical crosslinkers for hydrogels (superabsorbents), it is additionally necessary that the stabilizers used are nontoxic, since the stabilizers generally remain in the product and the end user is thus not endangered.

A multitude of stabilizers is known for (meth)acrylic acid and (meth)acrylic esters, referred to hereinbelow as (meth)acrylic acid/esters.

JP-A 60-72980 describes a stabilizer against free-radical polymerization or oxidative decomposition, comprising a chroman derivative of the formula (I) 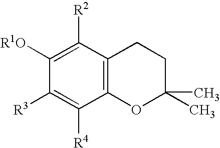
where R¹ is hydrogen, methyl, ethyl or acetyl, and R² to R⁴ are each hydrogen or methyl. Preference is given to 2,2,5,7,8-pentamethyl-6-chromanol, 2,2,5,7-tetramethyl-6-chromanol and 2,2,8-trimethyl-6-chromanol.

The stabilization of vinyl monomers is disclosed in general and the stabilization of acrylic acid in an inert atmosphere by way of example. Reference is further made to the similarity of such chromans to vitamin E. The examples show the superiority of 2,2,5,7,8-pentamethyl-6-chromanol compared to α-tocopherol in the stabilization of acrylic acid.

Indeed, chroman derivatives are frequently used as model systems for tocopherols or vitamin E (see, for example, J. Lars, G. Nilsson, H. Sievertsson, H. Selander, Acta Chemica Scandinavica, 22, (1968), 3160-3170. Especially 2,2,5,7,8-pentamethyl-6-chromanol is widely used as a model compound for α-tocopherol.

According to Römpp-Lexikon, vitamin E is a “collective term for fat-soluble, naturally occurring compounds having a chroman basic structure and a C₁₆ side chain”, i.e. tocopherols which are themselves defined as “3,4-dihydro-2H-benzopyran-6-ols (6-chromanols) substituted in the 2 position with a saturated or unsaturated 4,8,12-trimethyltridecyl radical”.

Some examples of such tocopherols are shown in formula (II) 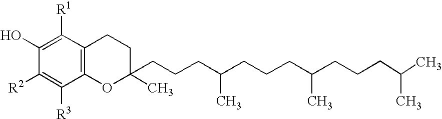
α-tocopherol where R¹, R², and R³═CH₃,
β-tocopherol where R¹ and R³═CH₃ and R²═H and
γ-tocopherol where R² and R³═CH₃ and R¹═H.

WO 90/07485 describes a process for preparing (meth)acrylic esters of polyhydric alcohols by esterifying (meth)acrylic acid with the appropriate alcohols, in which tocopherols are used as sterically hindered phenol compounds for stabilization and α-tocopherol is preferably used at least partly. The stabilization may also be effected in the presence of an oxygenous gas. It is unimportant whether the tocopherol is used in racemic or enantiomerically pure form.

A disadvantage of this process is the comparatively high price of tocopherol and a low effectiveness in relation thereto.

It is an object of the present invention to provide a process for preparing (meth)acrylic esters by which polymerization during the preparation can be more effectively reduced by a stabilizer than in the prior art, this stabilizer necessarily being nontoxic.

This object is achieved by a process for preparing (meth)acrylic esters from (meth)acrylic acid and at least one alcohol in the presence of at least one stabilizer, in which the stabilizer used is at least one 6-chromanol derivative of the formula (III), 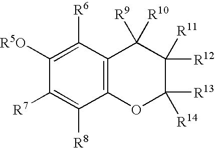
where
R⁵, R⁶, R⁷, R⁸, R⁹, R¹⁰, R¹¹, R¹², R¹³ and R¹⁴ are each independently hydrogen, C₁-C₄-alkyl, C₁-C₄-alkyloxy or C₆-C₁₂-aryl and
R⁵ is additionally C₁-C₄-alkylcarbonyl, C₁-C₄-alkyloxycarbonyl, C₆-C₁₂-arylcarbonyl or C₆-C₁₂-aryloxycarbonyl,
and the radicals mentioned may each optionally be interrupted by one or more oxygen atoms and/or sulfur atoms and/or one or more substituted or unsubstituted imino groups, or be substituted by functional groups, aryl, alkyl, aryloxy, alkyloxy, halogen, heteroatoms and/or heterocycles,
and R¹³ is additionally chlorine.

In this formula

C₁-C₄-alkyl optionally interrupted by one or more oxygen atoms and/or sulfur atoms and/or one or more substituted or unsubstituted imino groups, or substituted by functional groups, aryl, alkyl, aryloxy, alkyloxy, halogen, heteroatoms and/or heterocycles is, for example, methyl, ethyl, propyl, isopropyl, n-butyl, sec-butyl, tert-butyl, benzyl, 1-phenylethyl, 2-phenylethyl, α,α-dimethylbenzyl, benzhydryl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonethyl, 2-ethoxycarbonylethyl, 2-butoxycarbonylpropyl, 1,2-di(methoxycarbonyl)ethyl, 2-methoxyethyl, 2-ethoxyethyl, 2-butoxyethyl, diethoxymethyl, diethoxyethyl, 1,3-dioxolan-2-yl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyloxyethyl, chloromethyl, 2-chloroethyl, trichloromethyl, trifluoromethyl, 1,1-dimethyl-2-chloroethyl, 2-methoxyisopropyl, 2-ethoxyethyl, butylthiomethyl, 2-dodecylthioethyl, 2-phenylthioethyl, 2,2,2-trifluoroethyl, 2-hydroxyethyl, 2-hydroxypropyl, 3-hydroxypropyl, 4-hydroxybutyl, 2-aminoethyl, 2-aminopropyl, 3-aminopropyl, 4-aminobutyl, 2-methylaminoethyl, 2-methylaminopropyl, 3-methylaminopropyl, 4-methylaminobutyl, 2-dimethylaminoethyl, 2-dimethylaminopropyl, 3-dimethylaminopropyl, 4-dimethylaminobutyl, 2-hydroxy-2,2-dimethylethyl, 2-phenoxyethyl, 2-phenoxypropyl, 3-phenoxypropyl, 4-phenoxybutyl, 2-methoxyethyl, 2-methoxypropyl, 3-methoxypropyl, 4-methoxybutyl, 2-ethoxyethyl, 2-ethoxypropyl, 3-ethoxypropyl or 4-ethoxybutyl and

C₆-C₁₂-aryl optionally interrupted by one or more oxygen atoms and/or sulfur atoms and/or one or more substituted or unsubstituted imino groups, or substituted by functional groups, aryl, alkyl, aryloxy, alkyloxy, halogen, heteroatoms and/or heterocycles, is, for example, phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxymethylphenyl.

R⁵, R⁶, R⁷, R⁸, R⁹, R¹⁰, R¹¹, R¹², R¹³ and R¹⁴ are preferably each independently hydrogen or C₁-C₄-alkyl and more preferably hydrogen or methyl.

R⁵ is preferably hydrogen, C₁-C₄-alkyl or C₁-C₄-alkylcarbonyl, more preferably hydrogen or C₁-C₄-alkyl, and most preferably hydrogen, methyl or acetyl.

In particular, R⁵ and R⁹ to R¹² are each hydrogen, R⁶, R⁷ and R⁸ are each independently hydrogen or methyl, and R¹³ and R¹⁴ are each methyl.

R⁵ and R⁹ to R¹² are especially each hydrogen, R⁶, R⁷ and R⁸ especially each methyl, and R¹³ and R¹⁴ especially each methyl.

Preferred 6-chromanol derivatives are 2,2,5,7,8-pentamethyl-6-chromanol, 2,2,5,7-tetramethyl-6-chromanol, 2,2,5,8-tetramethyl-6-chromanol, 2,2,7,8-tetramethyl-6-chromanol, 2,2,5-trimethyl-6-chromanol, 2,2,7-trimethyl-6-chromanol and 2,2,8-trimethyl-6-chromanol, particularly preferred are 2,2,5,7,8-pentamethyl-6-chromanol, 2,2,5,7-tetramethyl-6-chromanol, 2,2,5,8-tetramethyl-6-chromanol and 2,2,7,8-tetramethyl-6-chromanol and very particularly preferred is 2,2,5,7,8-pentamethyl-6-chromanol.

According to the invention, 6-chromanols are used in processes for preparing an ester F of a polyalcohol A using at least one ethylenically unsaturated carboxylic acid B, comprising the steps of
a) reacting a polyalcohol A with at least one ethylenically unsaturated carboxylic acid B in the presence of at least one esterification catalyst C and at least one polymerization inhibitor D, and also, if appropriate, a solvent E which forms an azeotrope with water, to form an ester F,
b) if appropriate, removing at least a portion of the water formed in a) from the reaction mixture, b) being effected during and/or after a),
f) if appropriate, neutralizing the reaction mixture,
h) if a solvent E has been used, removing the solvent if appropriate by distillation, and/or
i) stripping with a gas inert under the reaction conditions.

The molar ratio of B to A is (per hydroxyl group to be esterified in the polyalcohol A) generally at least 1:1, preferably at least 1.05:1, more preferably at least 1.1:1, even more preferably at least 1.25:1 and in particular at least 1.5:1.

It will be appreciated that partial conversion of a polyalcohol A may also be sought, for example to prepare 2-hydroxyethyl (meth)acrylate or pentaerythrityl tri(meth)acrylate. To this end, correspondingly less carboxylic acid B is used, although random mixtures are generally obtained.

Useful polyalcohols A are compounds which have at least two hydroxyl functions (—OH), preferably at least three, more preferably from three to ten, even more preferably from three to six, and in particular from three to four.

The polyalcohols may be aliphatic, cycloaliphatic or aromatic, preferably aliphatic or cycloaliphatic and most preferably aliphatic, straight-chain or branched and optionally substituted with functional groups.

In general, the polyalcohols have from two to 50 carbon atoms and preferably from three to 40.

The molar mass of the polyalcohols which can be used is generally, unless stated otherwise, below 5000 g/mol, preferably below 2500 g/mol, more preferably below 1500 g/mol, even more preferably below 1000 g/mol and in particular below 800 g/mol.

Preferred polyalcohols A are polyols, functionalized polyols, alkoxylated polyols, sugar alcohols, partly alkoxylated sugar alcohols, polyetherols, polyesterols, at least partly alkoxylated polyesterols and at least partly hydrolyzed, alkoxylated polyesterols.

Examples of polyols are trimethylolbutane, trimethylolpropane, trimethylolethane, neopentyl glycol, neopentyl glycol hydroxypivalate, pentaerythritol, glycerol, 1,2-ethylene glycol, 1,2-propylene glycol, 2-ethyl-1,3-propanediol, 2-methyl-1,3-propanediol, hydroquinone, bisphenol A, bisphenol F, bisphenol B, 2,2-bis(4-hydroxycyclohexyl)propane, 1,1-, 1,2-, 1,3- and 1,4-cyclohexanedimethanol, 1,2-, 1,3- or 1,4-cyclohexanediol, but-2-ene-1,4-diol and but-2-yne-1,4-diol.

The polyols may also bear additional functionalities, for example ether functions (—O—), carboxyl functions (—COOH) or C₁-C₄-alkyloxycarbonyl functions (ester groups), and C₁-C₄-alkyl in this document means methyl, ethyl, isopropyl, n-propyl, n-butyl, isobutyl, sec-butyl or tert-butyl.

Examples of such functionalized polyols are ditrimethylolpropane, dipentaerythritol, dimethylolpropionic acid, dimethylolbutyric acid, trimethylolacetic acid, hydroxypivalic acid and the 2-hydroxyethyl or C₁-C₄-alkyl esters of these acids mentioned.

Preferred polyols are those of the formula (IV):

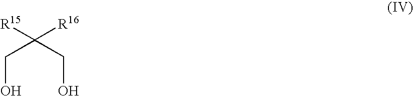

In this formula,
R¹⁵, R¹⁶ are each independently hydrogen, C₁-C₁₀-alkyl, preferably C₁-C₄-alkyl, C₁-C₁₀-hydroxyalkyl, preferably hydroxy-C₁-C₄-alkyl, carboxyl or C₁-C₄-alkyloxycarbonyl, preferably hydrogen, hydroxymethyl and C₁-C₄-alkyl, and more preferably hydroxymethyl and C₁-C₄-alkyl.

The alkyl radicals may each be straight-chain or branched.

Examples of R¹⁵ and R¹⁶ are hydrogen, methyl, ethyl, isopropyl, n-propyl, n-butyl, isobutyl, sec-butyl, tert-butyl, n-pentyl, n-hexyl, n-heptyl, n-octyl, n-decyl, hydroxymethyl, carboxyl, methoxycarbonyl, ethoxycarbonyl or n-butoxycarbonyl, preferably hydrogen, hydroxymethyl, methyl and ethyl, more preferably hydroxymethyl, methyl and ethyl.

Particularly preferred polyhydric alcohols of the formula (IV) are trimethylolbutane, trimethylolpropane, trimethylolethane, neopentyl glycol, pentaerythritol, 2-ethyl-1,3-propanediol, 2-methyl-1,3-propanediol, 1,3-propanediol, dimethylolpropionic acid, methyl dimethylolpropionate, ethyl dimethylolpropionate, dimethylolbutyric acid, methyl dimethylolbutyrate or ethyl dimethylolbutyrate; preference is given to neopentyl glycol, trimethylolpropane, pentaerythritol and dimethylolpropionic acid; very particular preference is given to neopentyl glycol, trimethylolpropane and pentaerythritol, and in particular to trimethylolpropane and pentaerythritol.

Examples of sugar alcohols are sorbitol, mannitol, maltitol, isomalt, diglycerol, threitol, erythritol, adonatol (ribitol), arabitol (Iyxitol), xylitol and dulcitol (galactitol).

Examples of polyetherols are polyTHF having a molar mass between 162 and 2000, preferably between 162 and 1458, more preferably between 162 and 1098, even more preferably between 162 and 738 and in particular between 162 and 378; poly-1,3-propanediol and poly-1,2-propanediol having a molar mass between 134 and 1178, preferably between 134 and 888, more preferably between 134 and 598 and even more preferably between 134 and 308; polyethylene glycol having a molar mass between 106 and 898, preferably between 106 and 458, more preferably from 106 to 400, even more preferably between 106 and 235, and in particular diethylene glycol, triethylene glycol and tetraethylene glycol.

Useful polyesterols are, for example, those which can be prepared by esterifying polycarboxylic acids, preferably dicarboxylic acids, with the abovementioned polyols.

The starting materials for such polyesterols are known to those skilled in the art. The polycarboxylic acids used may preferably be oxalic acid, maleic acid, fumaric acid, succinic acid, glutaric acid, adipic acid, sebacic acid, dodecanedioic acid, o-phthalic acid, isophthalic acid, terephthalic acid, trimellitic acid, azelaic acid, 1,4-cyclohexane-dicarboxylic acid or tetrahydrophthalic acid, their isomers and hydrogenation products, and also esterifiable derivatives such as anhydrides or dialkyl esters, for example C₁-C₄-alkyl esters, preferably methyl, ethyl or n-butyl esters, of the acids mentioned.

Useful carboxylic acids or lactones bearing hydroxyl groups include 4-hydroxybenzoic acid, 6-hydroxy-2-naphthoic acid, pivalolactone or ε-caprolactone. Useful polyols include the abovementioned polyfunctional alcohols, preferably neopentyl glycol, trimethylolpropane, trimethylolethane, pentaerythritol, dimethylolpropionic acid or dimethylolbutyric acid.

Preferred examples of such polyesterols are those of the formula (IVa-c) 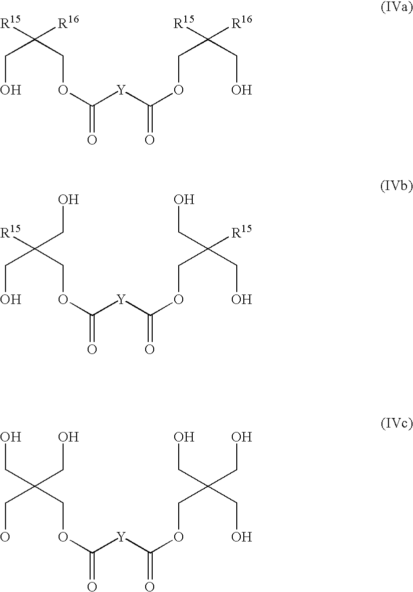
where
R¹⁵, R¹⁶ are each as defined above and
Y is a straight-chain or branched, optionally substituted alkylene group having from 2 to 20 carbon atoms or an optionally substituted cycloalkylene or arylene group having from 6 to 12 carbon atoms or a single bond.

Examples of Y are a single bond, methylene, 1,2-ethylene, 1,3-propylene, 1,4-butylene, 1,6-hexylene, 1,7-heptylene, 1,8-octylene, cis-1,2-ethenylene, trans-1,2-ethenylene, 1,2-, 1,3- or 1,4-phenylene, 1,2-cyclohex-1-enylene, 1,2-, 1,3- or 1,4-cyclohexylene, 4-carboxy-1,2-phenylene, 2-carboxy-1,4-phenylene or 1-carboxy-2,4-phenylene.

Preferred Y groups are 1,2-ethylene, 1,4-butylene and 1,2-, 1,3- or 1,4-phenylene.

It will be appreciated that as a result of the preparation, mixtures are generally present in which lower and higher oligomers may additionally be present.

Polyester (meth)acrylates may be prepared from (meth)acrylic acid, polycarboxylic acid and polyol in a plurality of stages or else in one stage, as described, for example, in EP-A 279 303.

Likewise useful as polyalcohols are alkoxylated polyols and polyesterols which are obtainable by reacting a polyol or polyesterol with at least one alkylene oxide.

It is also possible to prepare reaction mixtures comprising those compounds of the formula (V)
R¹⁷—(O(CH(R¹⁹)CH(R¹⁹)O)_{y}—C(═O)—R¹⁸)ₓ (V)
where
R¹⁷ is a polyfunctional, straight-chain or branched C₂-C₁₀-alkyl radical,
R¹⁸ is in each case independently a straight-chain or branched C₂-C₁₀-alkenyl radical,
R¹⁹ is in each case independently hydrogen or methyl,
x is in each case independently a positive integer of 2 or greater and
y is in each case independently, when x=2, a number from 3 to 8, and, when x=3 or greater, a number from 2 to 7.

The parent alcohol has the formula (Va)
R¹⁷—(O(CH(R¹⁹)CH(R¹⁹)O)_{y}—H)ₓ (Va)
where R¹⁷, R¹⁹, x and y are each as defined above.

The compounds of the formula (V) are polyhydric alcohols (Va) which generally have from 2 to 10 carbon atoms and which have been alkoxylated with between 2 and 8 alkylene oxide units per hydroxyl group and whose terminal hydroxyl group of each alkylene oxide chain has been esterified with an unsaturated carboxylic acid or ester thereof having from 2 to 10 carbon atoms. The starter alcohol is preferably a polyhydric alcohol having 3-6 carbon atoms and preferably bearing from 2 to 4 hydroxyl groups. The starter alcohol is more preferably trimethylolpropane, glycerol, pentaerythritol, 1,3-propanediol, propylene glycol, 1,4-butanediol or butylene glycol. Very particular preference is given to trimethylolpropane, glycerol and pentaerythritol as the starter alcohol.

Suitable alkylene oxides are, for example, ethylene oxide, propylene oxide, isobutylene oxide, vinyloxirane and/or styrene oxide.

The alkylene oxide chain may preferably be composed of ethylene oxide, propylene oxide and/or butylene oxide units. Such a chain may be composed of one species of an alkylene oxide or of a mixture of alkylene oxides. When a mixture is used, the different alkylene oxide units may be present randomly or as a block or blocks of individual species. The alkylene oxide is preferably ethylene oxide, propylene oxide or a mixture thereof, more preferably ethylene oxide or propylene oxide and most preferably ethylene oxide. One R¹⁹ radical per alkylene oxide unit is thus preferably hydrogen and the other methyl or hydrogen; more preferably, both R¹⁹ radicals are hydrogen.

The preferred number of alkylene oxide units in each chain is dependent upon the number of chains.

Frequently, these compounds of the formula (V) are present as a mixture of compounds which are described by this formula, and by-products of the preparation process.

Especially preferred among these compounds (V) are the compounds ethoxylated up to six times, more preferably up to four times and most preferably four times, per hydroxyl group, referred to hereinbelow as compounds (Vb). These have increased hydrolysis stability.

Also conceivable are those compounds (V) in which, when x=2, y may assume values of 0, 1 or 2, and, when x=3, y may assume values of 0 or 1.

Preferred examples of alkoxylated polyols are the alkoxylation products (VIa), (VIb) or (VIc) of polyols of the formula (IV) 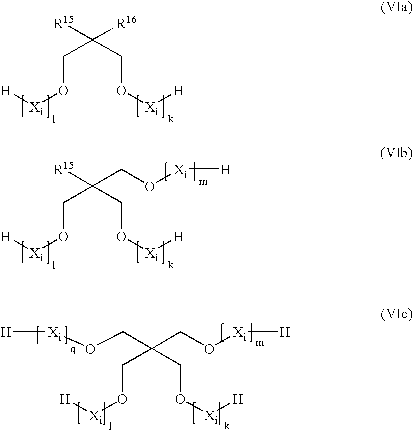
where
R¹⁵, R¹⁶ are each as defined above,
k, l, m, q are each independently an integer from 1 to 10, preferably from 1 to 5, more preferably from 3 to 5 and in particular 4, and
each Xᵢ, when i=from 1 to k, from 1 to 1, from 1 to m and from 1 to q, may independently be selected from the group of —CH₂—CH₂—O—, —CH₂—CH(CH₃)—O—, —CH(CH₃)—CH₂—O—, —CH₂—C(CH₃)₂—O—, —C(CH₃)₂—CH₂—O—, —CH₂—CHVin-O—, —CHVin-CH₂—O—, —CH₂—CHPh-O— and —CHPh-CH₂—O—, preferably from the group of —CH₂—CH₂—O—, —CH₂—CH(CH₃)—O— and —CH(CH₃)—CH₂—O—, and more preferably —CH₂—CH₂—O—,
   where Ph is phenyl and Vin is vinyl.

The alkoxylated polyol is preferably as singly to quintuply, more preferably triply to quintuply and most preferably quadruply ethoxylated, propoxylated or mixed ethoxylated and propoxylated, and especially exclusively ethoxylated, neopentyl glycol, trimethylolpropane, trimethylolethane or pentaerythritol.

Particular preference among these is given to those polyhydric alcohols of the formula (VIb).

Equally preferred is a singly to 20 tuply, preferably singly to decuply, more preferably doubly to decuply, even more preferably doubly to quintuply, in particular triply to quintuply and especially triply to quadruply alkoxylated, preferably ethoxylated, propoxylated or mixed ethoxylated-propoxylated and more preferably ethoxylated glycerol (as an exception, calculated here in mol of alkoxy group per mole of glycerol).

The degrees of alkoxylation specified each relate to the average degree of alkoxylation.

The number-average molar mass Mₙ of the alkoxylated polyols is preferably not more than 1000 g/mol, more preferably not more than 800 g/mol and most preferably not more than 550 g/mol.

The data on number-average and weight-average molecular weight Mₙ and M_{w} relate here to gel-permeation chromatography measurements, in which polystyrene was used as the standard and tetrahydrofuran as the eluent. The method is described in Analytiker Taschenbuch [Analyst's Handbook] vol. 4, pages 433 to 442, Berlin 1984.

Examples of alkoxylated sugar alcohols are those compounds which are obtainable from sugar alcohols, for example from the above-recited sugar alcohols, by alkoxylation, for example with the above-recited alkylene oxides, preferably with ethylene oxide and/or propylene oxide, and most preferably with ethylene oxide.

Examples thereof are
the tetrols recited which, on statistical average, per mole of sugar alcohol, have been 2-30tuply, preferably 2-20tuply, more preferably 3-10tuply and in particular 3-, 4-, 5-, 6-, 7- or 8tuply alkoxylated,
the pentols recited which, on statistical average, per mole of sugar alcohol, have been 3-35tuply, preferably 3-28tuply, more preferably 4-20tuply and in particular 4-, 5-, 6-, 7-, 8-, 9- or 10tuply alkoxylated,
higher sugar alcohols which, on statistical average, per mole of sugar alcohol, have been 4-50tuply, preferably 6-40tuply, more preferably 7-30tuply, even more preferably 8-20tuply and in particular 10-15tuply alkoxylated.

Preferred alkoxylated sugar alcohols are those in which at least one hydroxyl group of the sugar alcohol has not been alkoxylated.

Preferred examples of alkoxylated polyesterols are those of the formula (VIIa-c) 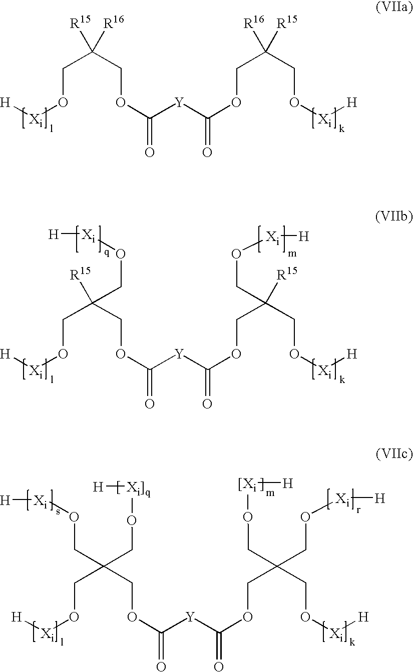
where
R¹⁵, R¹⁶, Y are each as defined above,
k, l, m, q, r, S are each independently an integer from 1 to 30, preferably from 1 to 20, more preferably from 1 to 10 and in particular from 1 to 5, and
each Xᵢ, when i=from 1 to k, from 1 to 1, from 1 to m, from 1 to q, from 1 to r and from 1 to s, may independently be selected from the group of —CH₂—CH₂—O—, —CH₂—CH(CH₃)—O—, —CH(CH₃)—CH₂—O—, —CH₂—C(CH₃)₂—O—, —C(CH₃)₂—CH₂—O—, —CH₂—CHVin-O—, —CHVin-CH₂—O—, —CH₂—CHPh-O— and —CHPh-CH₂—O—, preferably from the group of —CH₂—CH₂—O—, —CH₂—CH(CH₃)—O— and —CH(CH₃)—CH₂—O—, and more preferably —CH₂—CH₂—O—,
   where Ph is phenyl and Vin is vinyl.

The polyesterol is preferably an unalkoxylated or singly to decuply, more preferably doubly to quintuply ethoxylated, propoxylated or mixed ethoxylated and propoxylated neopentyl glycol, trimethylolpropane, trimethylolethane or pentaerythritol, each of which has been esterified with adipic acid, phthalic acid, terephthalic acid or isophthalic acid.

The reaction of the alcohols with an alkylene oxide is known per se to those skilled in the art. Possible embodiments can be found in Houben-Weyl, Methoden der Organischen Chemie, 4th edition, 1979, Thieme Verlag Stuttgart, ed. Heinz Kropf, volume 6/1a, part 1, pages 373 to 385.

When mixed alkoxylated alcohols are used, the different alkoxy groups present therein may be present in a molar ratio to one another of, for example, 0.05-20:1, preferably 0.1-10:1 and more preferably 0.2-5:1.

No particular demands are placed on the viscosity of the polyalcohols which can be used, except that they should be pumpable without any problem at a temperature up to approx. 80° C.; they should preferably have a viscosity below 1000 mPas, preferably below 800 mPas and most preferably below 500 mPas.

When the polyalcohols used in the reaction are trihydric or more highly functional polyalcohols, it may be sensible for their use as free-radical crosslinkers to convert the polyalcohols only partly. This means that, in the case of an n-hydric polyalcohol, only at least 2 of the n hydroxyl groups are reacted with the carboxylic acid B.

When n=3, the degree of reaction is at least 2, when n=4 at least 2, preferably at least 2.5 and more preferably at least 3, when n=5 or greater at least 2, preferably at least 3 and more preferably at least 4.

In such a case, the stoichiometric excess of carboxylic acid B to be used is calculated to the degree of conversion desired, and is thus, for example, 2/n times the above-specified molar excesses. It will be appreciated that the reaction may also be terminated, for example by cooling or dilution, when the desired degree of conversion has been attained.

Useful ethylenically unsaturated carboxylic acids B are those compounds which have at least one carboxyl group (—COOH), preferably one, and at least one, preferably one, ethylenically unsaturated group.

The useful carboxylic acids may be aliphatic, cycloaliphatic or aromatic, preferably aliphatic or cycloaliphatic and most preferably aliphatic, straight-chain or branched, and optionally substituted by functional groups.

In general, the carboxylic acids have from three to ten carbon atoms, preferably from three to five and more preferably from three to four.

Examples of ethylenically unsaturated carboxylic acids B are acrylic acid, methacrylic acid, ethyacrylic acid, maleic acid including its anhydride, fumaric acid, itaconic acid, citraconic acid, mesaconic acid, vinylacetic acid, allylacetic acid or crotonic acid.

Preferred carboxylic acids B are α,β-unsaturated carboxylic acids.

Particular preference is given to methacrylic acid and acrylic acid, referred to in this document as (meth)acrylic acid; very particular preference is given to acrylic acid.

In an alternative embodiment of the invention, the preparation of the (meth)acrylic esters may also be effected by a transesterification instead of an esterification. To this end, instead of a carboxylic acid B, a C₁-C₄-alkyl ester of a carboxylic acid B is used, i.e. a methyl, ethyl, n-propyl, isopropyl, n-butyl, sec-butyl, isobutyl or tert-butyl ester of a carboxylic acid B, preferably a methyl, ethyl or n-butyl ester, more preferably a methyl or ethyl ester and most preferably a methyl ester.

Useful esterification catalysts C are sulfuric acid, aryl- or alkylsulfonic acids or mixtures thereof. Examples of arylsulfonic acids are benzenesulfonic acid, para-toluenesulfonic acid or dodecylbenzenesulfonic acid; examples of alkylsulfonic acids are methanesulfonic acid, ethanesulfonic acid or trifluoromethanesulfonic acid. Strongly acidic ion exchangers or zeolites can also be used as esterification catalysts. Preference is given to sulfuric acid and sulfonic acids, particular preference to sulfuric acid and para-toluenesulfonic acid.

When the reaction is carried out as a transesterification instead of an esterification, the catalysts used for the preparation of (meth)acrylic esters by transesterification may, for example, be titanium alkoxides whose alkyl groups are C₁-C₄-alkyl radicals, for example tetramethyl, tetraethyl, tetraisopropyl, tetrapropyl, tetraisobutyl and tetrabutyl titanate (see, for example, EP-B1 298 867, EP-A2 960 877). Catalysts also proposed include titanium phenoxides (DE-A 200 86 18), metal chelate compounds of, for example, hafnium, titanium, zirconium or calcium, alkali metal and magnesium alkoxides, organic tin compounds, for example dimethyltin oxide or diphenyltin oxide, or calcium and lithium compounds, for example oxides, hydroxides, carbonates or halides.

For the process according to the invention, all transesterification catalysts described in the prior art may be used, preferably titanium alkoxides, magnesium alkoxides or aluminum alkoxides, more preferably titanium alkoxides and in particular titanium tetramethoxide, tetraethoxide, tetraisopropoxide and tetra-n-butoxide.

It is essential to the invention that at least one chromanol derivative of the formula (III) is present as a polymerization inhibitor D during the esterification or transesterification. It will be appreciated that 2 or more may also be present; preference is given to one chromanol derivative being present.

Polymerization inhibitors D which can be used additionally thereto if appropriate are, for example, phenols such as

alkylphenols, for example o-, m- or p-cresol (methylphenol), 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tert-butylphenol, 2-methyl-4-tert-butylphenol, 4-tert-butyl-2,6-dimethylphenol, or 2,2′-methylenebis(6-tert-butyl-4-methylphenol), 4,4′-oxydiphenyl, 3,4-(methylenedioxy)diphenol (sesamol), 3,4-dimethylphenol, hydroquinone, pyrocatechol (1,2-dihydroxybenzene), 2-(1′-methylcyclohex-1′-yl)-4,6-dimethylphenol, 2- or 4-(1′-phenyleth-1′-yl)phenol, 2-tert-butyl-6-methylphenol, 2,4,6-tris-tert-butylphenol, 2,6-di-tert-butylphenol, 2,4-di-tert-butylphenol, 4-tert-butylphenol, nonylphenol [11066-49-2], octylphenol [140-66-9], 2,6-dimethylphenol, bisphenol A, bisphenol F, bisphenol B, bisphenol C, bisphenol S, 3,3′,5,5′-tetrabromobisphenol A, 2,6-di-tert-butyl-p-cresol, Koresin® from BASF AG, methyl 3,5-di-tert-butyl-4-hydroxybenzoate, 4-tert-butylpyrocatechol, 2-hydroxybenzyl alcohol, 2-methoxy-4-methylphenol, 2,3,6-trimethylphenol, 2,4,5-trimethylphenol, 2,4,6-trimethylphenol, 2-isopropylphenol, 4-isopropylphenol, 6-isopropyl-m-cresol, n-octadecyl β-(3,5-di-tert-butyl-4-hydroxyphenyl)-propionate, 1,1,3-tris(2-methyl-4-hydroxy-5-tert-butylphenyl)butane, 1,3,5-trimethyl-2,4,6-tris(3,5-di-tert-butyl-4-hydroxybenzyl)benzene, 1,3,5-tris(3,5-di-tert-butyl-4-hydroxybenzyl)isocyanurate, 1,3,5-tris(3,5-di-tert-butyl-4-hydroxyphenyl)propionyl-oxyethyl isocyanurate, 1,3,5-tris(2,6-dimethyl-3-hydroxy-4-tert-butylbenzyl)isocyanurate or pentaerythrityl tetrakis[β-(3,5-di-tert-butyl-4-hydroxyphenyl)propionate], 2,6-di-tert-butyl-4-dimethylaminomethylphenol, 6-iso-butyl-2,4-dinitrophenol, 6-sec-butyl-2,4-dinitrophenol, Irganox® 565, 1141, 1192, 1222 and 1425 from Ciba Spezialitätenchemie, octadecyl 3-(3′,5′-di-tert-butyl-4′-hydroxyphenyl)propionate, hexadecyl 3-(3′,5′-di-tert-butyl-4′-hydroxyphenyl)propionate, octyl 3-(3′,5′-di-tert-butyl-4′-hydroxyphenyl)propionate, 3-thia-1,5-pentanediyl bis[(3′,5′-di-tert-butyl-4′-hydroxyphenyl)propionate], 4,8-dioxa-1,11-undecanediyl bis[(3′,5′-di-tert-butyl-4′-hydroxyphenyl)propionate], 4,8-dioxa-1,11-undecanediyl bis[(3′-tert-butyl-4′-hydroxy-5′-methylphenyl)propionate], 1,9-nonanediyl bis[(3′,5′-di-tert-butyl-4′-hydroxyphenyl)propionate], 1,7-heptanediamine bis[3-(3′,5′-di-tert-butyl-4′-hydroxyphenyl)propionamide], 1,1-methanediamine bis[3-(3′,5′-di-tert-butyl-4′-hydroxyphenyl)propionamide], 3-(3′,5′-di-tert-butyl-4′-hydroxyphenyl)propionic acid hydrazide, 3-(3′,5′-di-methyl-4′-hydroxyphenyl)propionic acid hydrazide, bis(3-tert-butyl-5-ethyl-2-hydroxyphen-1-yl)methane, bis(3,5-di-tert-butyl-4-hydroxyphen-1-yl)methane, bis[3-(1′-methylcyclohex-1′-yl)-5-methyl-2-hydroxyphen-1-yl]methane, bis(3-tert-butyl-2-hydroxy-5-methylphen-1-yl)methane, 1,1-bis(5-tert-butyl-4-hydroxy-2-methylphen-1-yl)ethane, bis(5-tert-butyl-4-hydroxy-2-methylphen-1-yl)sulfide, bis(3-tert-butyl-2-hydroxy-5-methylphen-1-yl) sulfide, 1,1-bis(3,4-dimethyl-2-hydroxyphen-1-yl)-2-methylpropane, 1,1-bis(5-tert-butyl-3-methyl-2-hydroxyphen-1-yl)butane, 1,3,5-tris-[1′-(3″,5″-di-tert-butyl-4″-hydroxyphen-1″-yl)meth-1′-yl]-2,4,6-trimethylbenzene, 1,1,4-tris(5′-tert-butyl-4′-hydroxy-2′-methylphen-1′-yl)butane,

aminophenols, for example para-aminophenol, 3-diethylaminophenol,

nitrosophenols, for example para-nitrosophenol, p-nitroso-o-cresol,

alkoxyphenols, for example 2-methoxyphenol (guaiacol, pyrocatechol monomethyl ether), 2-ethoxyphenol, 2-isopropoxyphenol, 4-methoxyphenol (hydroquinone monomethyl ether), mono- or di-tert-butyl-4-methoxyphenol, 3,5-di-tert-butyl-4-hydroxyanisole, 3-hydroxy-4-methoxybenzyl alcohol, 2,5-dimethoxy-4-hydroxybenzyl alcohol (syringa alcohol), 4-hydroxy-3-methoxybenzaldehyde (vanillin), 4-hydroxy-3-ethoxybenzaldehyde (ethylvanillin), 3-hydroxy-4-methoxybenzaldehyde (isovanillin), 1-(4-hydroxy-3-methoxyphenyl)ethanone (acetovanillone), eugenol, dihydroeugenol, isoeugenol,

tocopherols, for example α-, β-, γ-, δ- and ε-tocopherol, tocol, α-tocopherol-hydroquinone,

and also 2,3-dihydro-2,2-dimethyl-7-hydroxybenzofuran (2,2-dimethyl-7-hydroxy-coumarin), Trolox®), gallic acid, ferulic acid, cinnamic acid and derivatives thereof,

quinones and hydroquinones,

hydroquinone or hydroquinone monomethyl ether, 2,5-di-tert-butylhydroquinone, 2-methyl-p-hydroquinone, 2,3-dimethylhydroquinone, trimethylhydroquinone, 4-methylpyrocatechol, tert-butylhydroquinone, 3-methylpyrocatechol, benzoquinone, 2-methyl-p-hydroquinone, 2,3-dimethylhydroquinone, trimethylhydroquinone, 3-methylpyrocatechol, 4-methylpyrocatechol, tert-butylhydroquinone, 4-ethoxyphenol, 4-butoxyphenol, hydroquinone monobenzyl ether, p-phenoxyphenol, 2-methylhydroquinone, 2,5-di-tert-butylhydroquinone, tetramethyl-p-benzoquinone, diethyl 1,4-cyclohexanedione-2,5-dicarboxylate, phenyl-p-benzoquinone, 2,5-dimethyl-3-benzyl-p-benzoquinone, 2-isopropyl-5-methyl-p-benzoquinone (thymoquinone), 2,6-diisopropyl-p-benzoquinone, 2,5-dimethyl-3-hydroxy-p-benzoquinone, 2,5-dihydroxy-p-benzoquinone, embelin, tetrahydroxy-p-benzoquinone, 2,5-dimethoxy-1,4-benzoquinone, 2-amino-5-methyl-p-benzoquinone, 2,5-bisphenylamino-1,4-benzoquinone, 5,8-dihydroxy-1,4-naphthoquinone, 2-anilino-1,4-naphthoquinone, anthraquinone, N ,N-di-methylindoaniline, N,N-diphenyl-p-benzoquinonediimine, 1,4-benzoquinone dioxime, coerulignone, 3,3′-di-tert-butyl-5,5′-dimethyldiphenoquinone, p-rosolic acid (aurine), 2,6-di-tert-butyl-4-benzylidenebenzoquinone, 2,5-di-tert-amylhydroquinone,

N-oxyls,

4-hydroxy-2,2,6,6-tetramethylpiperidine N-oxyl, 4-oxo-2,2,6,6-tetramethylpiperidine N-oxyl, 4-acetoxy-2,2,6,6-tetramethylpiperidine N-oxyl, 2,2,6,6-tetramethylpiperidine N-oxyl, 4,4′,4″-tris(2,2,6,6-tetramethylpiperidinyloxy)phosphite, 3-oxo-2,2,5,5-tetramethylpyrrolidine N-oxyl, 1-oxyl-2,2,6,6-tetramethyl-4-methoxypiperidine, 1-oxyl-2,2,6,6-tetramethyl-4-trimethylsilyloxypiperidine, 1-oxyl-2,2,6,6-tetramethylpiperidin-4-yl 2-ethylhexanoate, 1-oxyl-2,2,6,6-tetramethylpiperidin-4-yl stearate, 1-oxyl-2,2,6,6-tetramethylpiperidin-4-yl benzoate, 1-oxyl-2,2,6,6-tetramethylpiperidin-4-yl (4-tert-butyl)benzoate, bis(1-oxyl-2,2,6,6-tetramethylpiperidin-4-yl)succinate, bis(1-oxyl-2,2,6,6-tetramethylpiperidin-4-yl)adipate, bis(1-oxyl-2,2,6,6-tetramethyl-4-piperidinyl) 1,10-decanedioate, bis(1-oxyl-2,2,6,6-tetramethyl-4-piperidinyl)n-butylmalonate, bis(1-oxyl-2,2,6,6-tetramethyl-4-piperidinyl)phthalate, bis(1-oxyl-2,2,6,6-tetramethyl-4-piperidinyl)isophthalate, bis(1-oxyl-2,2,6,6-tetramethyl-4-piperidinyl)terephthalate, bis(1-oxyl-2,2,6,6-tetramethyl-4-piperidinyl)hexahydroterephthalate, N ,N′-bis(1-oxyl-2,2,6,6-tetramethyl-4-piperidinyl)adipamide, N-(1-oxyl-2,2,6,6-tetramethyl-4-piperidinyl)caprolactam, N-(1-oxyl-2,2,6,6-tetramethyl-4-piperidinyl)dodecyl-succinimide, 2,4,6-tris[N-butyl-N-(1-oxyl-2,2,6,6-tetramethyl-4-piperidinyl]triazine, N,N′-bis(1-oxyl-2,2,6,6-tetramethyl-4-piperidinyl)-N,N′-bisformyl-1,6-diaminohexane, 4,4′-ethylenebis(1-oxyl-2,2,6,6-tetramethyl-3-piperazinone),

aromatic amines, phenylenediamines,

N,N-diphenylamine, N-nitrosodiphenylamine, nitrosodiethylaniline, N,N′-dialkyl-para-phenylenediamine, wherein the alkyl radicals can be the same or different and may each independently contain from 1 to 4 carbon atoms and be straight-chain or branched, for example N,N′-di-iso-butyl-p-phenylenediamine, N,N′-di-iso-propyl-p-phenylenediamine, Irganox 5057 from Ciba Spezialitätenchemie, N,N′-di-iso-butyl-p-phenylenediamine, N,N′-di-iso-propyl-p-phenylenediamine, p-phenylenediamine, N-phenyl-p-phenylenediamine, N,N′-diphenyl-p-phenylenediamine, N-isopropyl-N-phenyl-p-phenylenediamine, N,N′-di-sec-butyl-p-phenylenediamine (Kerobit® BPD from BASF AG), N-phenyl-N′-isopropyl-p-phenylenediamine (Vulkanox® 4010 from Bayer AG), N-(1,3-dimethylbutyl)-N′-phenyl-p-phenylenediamine, N-phenyl-2-naphthylamine, iminodibenzyl, N,N′-diphenylbenzidine, N-phenyltetraaniline, acridone, 3-hydroxydiphenylamine, 4-hydroxydiphenylamine,

sulfonamides,

sulfonamides effective as stabilizers are described, for example, in DE-A 10258329

oximes,

oximes may be, for example, aldoximes, ketoximes or amidoximes, described, for example, in DE 10139767, preferably diethyl ketoxime, acetone oxime, methyl ethyl ketoxime, cyclohexanone oxime or other aliphatic oximes, or their reaction products with alkyl transfer reagents,

hydroxylamines,

N,N-diethylhydroxylamine,

urea derivatives,

urea or thiourea,

phosphorus compounds,

triphenylphosphine, triphenyl phosphite, hypophosphorous acid or triethyl phosphite,

sulfur compounds,

diphenyl sulfide, phenothiazine, ovothiols, further sulfur-containing natural substances such as cysteine,

metal salts,

copper or metal salts, for example copper chloride, copper dithiocarbamate, copper sulfate, copper salicylate, copper acetate, manganese chloride, manganese dithiocarbamate, manganese sulfate, manganese salicylate, manganese acetate, cerium chloride, cerium dithiocarbamate, cerium sulfate, cerium salicylate, cerium acetate, nickel chloride, nickel dithiocarbamate, nickel sulfate, nickel salicylate, nickel acetate, chromium chloride, chromium dithiocarbamate, chromium sulfate, chromium salicylate or chromium acetate.

Preference is given to the phenols and quinones mentioned, particular preference to hydroquinone, hydroquinone monomethyl ether, 2-tert-butyl-4-methylphenol, 6-tert-butyl-2,4-dimethylphenol, 2,6-di-tert-butyl-4-methylphenol, 2,4-di-tert-butylphenol, triphenyl phosphite, hypophosphorous acid, CuCl₂ and guaiacol, and very particular preference to hydroquinone and hydroquinone monomethyl ether.

Among the additional stabilizers recited, preference is given to those which are aerobic, i.e. those which require the presence of oxygen to fully develop their inhibiting action.

The present invention further relates to stabilizer mixtures comprising at least one 6-chromanol derivative of the formula (III) and at least one stabilizer selected from the group consisting of phenols, quinones and hydroquinones, N-oxyls, aromatic amines, phenylenediamines, sulfonamides, oximes, hydroxylamines, urea derivatives, phosphorus compounds, sulfur compounds and metal salts.

Preferred stabilizer mixtures comprise at least one 6-chromanol derivative of the formula (III) and at least one stabilizer selected from the group consisting of phenols, hydroquinones, N-oxyls, and sulfur compounds.

Particularly preferred stabilizer mixtures comprise at least one 6-chromanol derivative of the formula (III) and at least one stabilizer selected from the group consisting of phenols and sulfur compounds.

Very particular preference is given to stabilizer mixtures comprising at least one 6-chromanol derivative of the formula (III) and at least one stabilizer selected from the group consisting of phenothiazine, hydroquinone, hydroquinone monomethyl ether and hypophosphorous acid.

In a preferred embodiment, the stabilization is further supported by carrying out the reaction in an oxygenous gas, preferably air or a mixture of air and nitrogen (lean air).

To this end, preference is given to continuously purging the reaction zone and/or the heat exchangers installed in the plant, for example distillation units or reactors, with a gas or gas mixture inert under the reaction conditions, for example nitrogen, air, nitrogen-oxygen mixtures, argon, helium, carbon dioxide or monoxide, preferably air or air-nitrogen mixtures, especially those having an oxygen content of from 0.1 up to 15% by volume, preferably from 0.5 up to 10% by volume and more preferably those air-nitrogen mixtures having an oxygen content of from 1 to 5% by volume. Preference is given to passing the purge gas along the heat exchanger surfaces present, more preferably in a forced or natural circulation evaporator which is present.

To this end, the purge gas is metered under pressure or volume control through a suitable unrestricted feed apparatus known per se in the vicinity of a heat exchanger surface present, so that the preferably continuous purge gas stream is conducted along the heat exchanger surface in cocurrent or countercurrent to the liquid.

Useful solvents E are particularly those which are suitable for azeotropically removing the water of reaction, if desired, in particular aliphatic, cycloaliphatic and aromatic hydrocarbons or mixtures thereof.

Preference is given to using n-pentane, n-hexane, n-heptane, cyclohexane, methylcyclohexane, benzene, toluene or xylene. Particular preference is given to cyclohexane, methylcyclohexane and toluene.

For the esterification, the preparation and/or workup processes, known to those skilled in the art, of polyhydric alcohols may be employed, for example those described in DE-A 199 41 136, DE-A 38 43 843, DE-A 38 43 854, DE-A 199 37 911, DE-A 199 29 258, EP-A 331 845, EP 554 651 or U.S. Pat. No. 4,187,383.

In general, the esterification may be carried out as follows:

the esterification reactor consists of a stirred reactor, preferably of a reactor having a circulation evaporator and an attached distillation unit having a condenser and a phase separation vessel.

The reactor may, for example, be a reactor having jacket heating or/and internal heating coils. Preference is given to using a reactor having an external heat exchanger and natural or forced circulation, i.e. using a pump, more preferably natural circulation in which the circulation stream is brought about without mechanical aids.

It will be appreciated that the reaction may also be carried out in a plurality of reaction zones, for example a reactor battery composed of from two to four, preferably from two to three, reactors.

Suitable circulation evaporators are known to those skilled in the art and are described, for example, in R. Billet, Verdampfertechnik [Evaporator Technology], HTB-Verlag, Bibliographisches Institut Mannheim, 1965, 53. Examples of circulation evaporators are tube bundle heat exchangers, plate heat exchangers, etc.

It will be appreciated that a plurality of heat exchangers may also be present in the circulation system.

The design of the distillation unit is known per se. It may be a simple distillation which is, if appropriate, equipped with a splash guard, or a rectification column. Useful column internals are in principle all common internals, for example trays, structured packings and/or random packings. Of the trays, preference is given to bubble-cap trays, sieve trays, valve trays, Thormann trays and/or dual-flow trays; of the random packings, preference is given to those having rings, spirals, saddles or braids.

In general, from 5 to 20 theoretical plates are sufficient.

The condenser and the separation vessel are of conventional design.

Carboxylic acid B and polyalcohol A are used in the esterification a) generally in a molar excess, as specified above, based on the hydroxyl groups of the alcohol.

Useful esterification catalysts C are those recited above.

They are generally used in an amount of 0.1-5% by weight, based on the esterification mixture, preferably 0.5-5%, more preferably 14% and most preferably 24% by weight.

If necessary, the esterification catalyst may be removed from the reaction mixture with the aid of an ion exchanger. The ion exchanger may be added directly to the reaction mixture and subsequently filtered off, or the reaction mixture may be passed through an ion exchanger bed.

Preference is given to leaving the esterification catalyst in the reaction mixture. However, when the catalyst is an ion exchanger, preference is given to removing it, for example by filtration.

To further promote the stabilization, an oxygenous gas, preferably air or a mixture of air and nitrogen (lean air) may be present.

This oxygenous gas is preferably metered into the bottom region of a column and/or into a circulation evaporator and/or passed through the reaction mixture and/or over it.

The inventive polymerization inhibitor D or a mixture comprising it (as detailed above) is generally used in a total amount of 0.01-5% by weight, based on the esterification mixture, preferably 0.02-3%, more preferably 0.05-2% by weight, even more preferably from 0.1 to 1% and in particular from 0.3 to 1% by weight.

The polymerization inhibitor (mixture) D may be used, for example, in the form of an aqueous solution or in the form of a solution in a reactant or product or another suitable solvent, for example those mentioned under E.

When the reaction is carried out as a transesterification, the transesterification is generally carried out in a similar manner to the esterification described. In the context of this document, transesterification is to be regarded as similar to esterification, even when this is not stated explicitly. A significant difference is that, in contrast to the esterification, the C₁-C₄-alcohol released has to be removed for better shifting of the equilibrium. This may be effected, for example, by distillation, if appropriate through a column placed on top of or attached to the reactor, and/or by stripping, i.e. by passing a preferably oxygenous gas through the reaction mixture.
b) The water of reaction formed in the reaction may be distilled off during or after the esterification a), and this operation may be promoted by a solvent which forms an azeotrope with water.

Suitable solvents E for azeotropically removing the water of reaction, if desired, are the above-recited compounds.

Preference is given to carrying out the esterification in the presence of a solvent.

The amount of solvent used is 10-200% by weight, preferably 20-100% by weight, more preferably from 30 to 100% by weight, based on the sum of polyalcohol and carboxylic acid B.

However, conduct without azeotroping agent is also conceivable, as described, for example, in DE-A138 43 854, col. 2, line 18 to col. 4, line 45, but, in contrast thereto, with the abovementioned stabilizers.

If the water present in the reaction mixture is not removed by an azeotrope-forming solvent, it is possible to remove it by stripping with an inert gas, preferably an oxygenous gas, more preferably with air or lean air, for example as described in DE-A 38 43 843.

The reaction temperature of the esterification a) is generally 40-160° C., preferably 60-140° C. and more preferably 80-120° C. The temperature may remain constant or rise in the course of the reaction; preference is given to raising it in the course of the reaction. In this case, the end temperature of the esterification is 5-30° C. higher than the starting temperature. The temperature of the esterification may be determined and controlled by varying the solvent concentration in the reaction mixture, as described in DE-A 199 41 136 and DE-A 100 63 175.

If a solvent is used, it may be distilled off from the reaction mixture via the distillation unit placed on top of the reactor.

The distillate may, as desired, be removed, or, after condensation, be conducted into a phase separation apparatus. The thus obtained aqueous phase is generally discharged; the organic phase may be conducted into the distillation unit as reflux, and/or be passed directly into the reaction zone, and/or be conducted into a circulation evaporator, as described in DE-A 100 63 175.

In the case of use as reflux, the organic phase, as described in DE-A 199 41 136, may be used to control the temperature in the esterification.

The esterification a) may be carried out at ambient pressure or else under elevated pressure or reduced pressure; preference is given to working at atmospheric pressure.

The reaction time is generally 2-20 hours, preferably 4-15 hours and more preferably from 7 to 12 hours.

The sequence in which the individual reaction components are added is not essential to the invention. It is possible to initially charge all components in a mixture and subsequently heat them, or one or more components may only partly be initially charged, if at all, and only added after the heating.

The composition of the carboxylic acid B which can be used is not restricted and may, in the case of crude (meth)acrylic acid, for example, have the following components:

| | | |
|---|---|---|
| (Meth)acrylic acid | 90-99.9% | by weight |
| Acetic acid | 0.05-3% | by weight |
| Propionic acid | 0.01-1% | by weight |
| Diacrylic acid | 0.01-5% | by weight |
| Water | 0.05-5% | by weight |
| Carbonyls | 0.01-0.3% | by weight |
| Inhibitors | 0.01-0.1% | by weight |
| Maleic acid/anhydride | 0.001-0.5% | by weight |

The crude (meth)acrylic acid used is generally stabilized with 200-600 ppm of phenothiazine or other stabilizers in amounts which enable comparable stabilization. The term carbonyls refers here, for example, to acetone and lower aldehydes, for example formaldehyde, acetaldehyde, crotonaldehyde, acrolein, 2- and 3-furfural and benzaldehyde.

Crude (meth)acrylic acid refers here to the (meth)acrylic acid-containing mixture which is obtained after absorption of the reaction gases of the propane/propene/acrolein or isobutane/isobutene/methacrolein oxidation in an absorbent and subsequent removal of the absorbent, or which is obtained by fractional condensation of the reaction gases.

It will be appreciated that it is also possible to use pure (meth)acrylic acid having, for example, the following purity:

| | | |
|---|---|---|
| (Meth)acrylic acid | 99.7-99.99% | by weight |
| Acetic acid | 50-1000 | ppm by weight |
| Propionic acid | 10-500 | ppm by weight |
| Diacrylic acid | 10-500 | ppm by weight |
| Water | 50-1000 | ppm by weight |
| Carbonyls | 1-500 | ppm by weight |
| Inhibitors | 1-300 | ppm by weight |
| Maleic acid/anhydride | 1-200 | ppm by weight |

The crude (meth)acrylic acid used is generally stabilized with 100-300 ppm of hydroquinone monomethyl ether or other storage stabilizers in amounts which enable comparable stabilization.

Pure or prepurified (meth)acrylic acid generally refers to (meth)acrylic acid whose purity is at least 99.5% by weight and which is substantially free of the aldehydic, other carbonylic and high-boiling components.

The aqueous phase, distilled off during the esterification, of the condensate which is removed via the column placed on top, if provided and may generally contain 0.1-10% by weight of carboxylic acid B, for example (meth)acrylic acid, is removed and discharged. Advantageously, the carboxylic acid present therein, for example (meth)acrylic acid, may be extracted with an extractant, preferably the solvent used if desired in the esterification, for example with cyclohexane, at a temperature between 10 and 40° C. and a ratio of aqueous phase to extractant of 1:5-30, preferably 1:10-20, and recycled into the esterification.

To further support the circulation, an inert gas, preferably an oxygenous gas, more preferably air or a mixture of air and nitrogen (lean air) may be passed into the circulation, through or over the reaction mixture, for example in amounts of 0.1-1 m³/m³h, preferably 0.2-0.8 m³/m³h and more preferably 0.3-0.7 m³/m³h, based on the volume of the reaction mixture.

The progress of the esterification a) may be monitored by monitoring the amount of water discharged and/or the reduction in the carboxylic acid concentration in the reactor.

The reaction may be ended, for example, as soon as 90% of the amount of water to be expected theoretically has been discharged by the solvent, preferably at least 95% and more preferably at least 98%. It will be appreciated that it is also possible to terminate the reaction even at lower partial conversions.

In general, the reaction is ended when the acid number to DIN EN 3682 of the reaction mixture goes below 80 mg KOH/g of reaction mixture, preferably goes below 60, more preferably 50 and most preferably 40 mg KOH/g.

The end of the reaction may be detected, for example, by substantially no further water of reaction being removed via the azeotroping agent. When carboxylic acid B is discharged together with the water of reaction, its proportion can be determined, for example, by back titration of an aliquot of the aqueous phase.

It is possible to dispense with removal of the water of reaction when the carboxylic acid B is used in a high stoichiometric excess, for example of at least 1.5:1, preferably at least 2.5:1 and most preferably at least 5:1. In this case, a substantial portion of the amount of water formed remains in the reaction mixture. During or after the reaction, only that proportion of water which is determined by the volatility at the temperature employed is removed from the reaction mixture and, furthermore, no measures are carried out to remove the water of reaction formed. For example, at least 10% by weight of the water of reaction formed may remain in the reaction mixture, preferably at least 20% by weight, more preferably at least 30% by weight, even more preferably at least 40% by weight and in particular at least 50% by weight.
c) On completion of the esterification, the reaction mixture may be cooled to a temperature of from 10 to 30° C. in a customary manner and, if appropriate, be adjusted to any target ester concentration by addition of solvent which may be the same solvent as any solvent used to azeotropically remove water or another solvent.

In a further embodiment, the reaction may be stopped using a suitable diluent G and diluted to a concentration of, for example, 10-90% by weight, preferably 20-80% by weight, more preferably from 20 to 60%, even more preferably from 30 to 50% and in particular approx. 40%, for example in order to reduce the viscosity.

It is important that a substantially homogeneous solution is formed after dilution.

This is preferably not done until relatively shortly before use in the preparation of the hydrogel, for example not more than 24 hours beforehand, preferably not more than 20 hours, more preferably not more than 12 hours, even more preferably not more than 6 hours and in particular not more than 3 hours beforehand.

The diluent G is selected from the group consisting of water, a mixture of water with one or more organic solvents having unlimited solubility in water or a mixture of water with one or more simple or polyfunctional alcohols, for example methanol and glycerol. The alcohols preferably bear 1, 2 or 3 hydroxyl groups and preferably have between 1 and 10, in particular up to 4 carbon atoms. Preference is given to primary and secondary alcohols.

Preferred alcohols are methanol, ethanol, isopropanol, ethylene glycol, 1,2-propanediol or 1,3-propanediol.
d) If necessary, the reaction mixture may be subjected to decolorization, for example by treatment with activated carbon or metal oxides, for example alumina, silica, magnesium oxide, zirconium oxide, boron oxide or mixtures thereof, in amounts of, for example, 0.1-50% by weight, preferably from 0.5 to 25% by weight, more preferably 1-10% by weight, at temperatures of, for example, from 10 to 100° C., preferably from 20 to 80° C. and more preferably from 30 to 60° C.

This may be effected by adding the pulverulent or granular decolorizing agent to the reaction mixture and subsequently filtering or by passing the reaction mixture over a bed of the decolorizing agent in the form of any suitable shaped bodies.

The decolorization of the reaction mixture can be effected at any point in the workup process, for example at the stage of the crude reaction mixture or after any prewash, neutralization, wash or solvent removal effected.

The reaction mixture may also be subjected to a prewash e) and/or a neutralization f and/or a postwash g) preferably merely to a neutralization f. If desired, the sequence of neutralization f) and prewash e) may be interchanged.

From the aqueous phase of the washes e) and g) and/or neutralization f), it is possible to at least partly recover carboxylic acid B present, for example (meth)acrylic acid, and/or catalyst C by acidifying and extracting with a solvent, and use them anew.

For the prewash or postwash e) or g), the reaction mixture is treated in a wash apparatus with a wash liquid, for example water or a 5-30% by weight, preferably 5-20%, more preferably 5-15% by weight sodium chloride, potassium chloride, ammonium chloride, sodium sulfate or ammonium sulfate solution, preferably water or sodium chloride solution.

The reaction mixture:wash liquid ratio is generally 1:0.1-1, preferably 1:0.2-0.8, more preferably 1:0.3-0.7.

The wash or neutralization may be carried out, for example, in a stirred vessel or in other conventional apparatus, for example in a column or mixer-settler apparatus.

From a process technology point of view, a wash or neutralization in the process described may be effected using all extraction and washing processes and apparatus known per se, for example those which are described in Ullmann's Encyclopedia of Industrial Chemistry, 6th ed, 1999 Electronic Release, chapter: Liquid-Liquid Extraction-Apparatus. For example, these may be single-stage or multistage, preferably single-stage, extractions, and may also be those in cocurrent or countercurrent mode, preferably countercurrent mode.

Preference is given to using columns having sieve trays or random packings, stirred tanks or mixer-settler apparatus, and also pulsed columns or those having rotating internals.

The prewash e) is preferably used when metal salts, more preferably copper or copper salts, are also used as inhibitors.

A postwash g) may be advantageous to remove base or salt traces from the reaction mixture neutralized in f).

For neutralization f), the reaction mixture which may have been prewashed and may still contain small amounts of catalyst and the majority of excess carboxylic acid, for example (meth)acrylic acid, may be neutralized with a 5-25%, preferably 5-20%, more preferably 5-15% by weight aqueous solution of a base, for example alkali metal or alkaline earth metal oxides, hydroxides, carbonates or hydrogencarbonates, preferably sodium hydroxide solution, potassium hydroxide solution, sodium hydrogencarbonate, sodium carbonate, potassium hydrogencarbonate, calcium hydroxide, milk of lime, ammonia, aqueous ammonia or potassium carbonate, to which may, if desired, be added 5-15% by weight of sodium chloride, potassium chloride, ammonium chloride or ammonium sulfate, more preferably with sodium hydroxide solution or a solution of sodium hydroxide and sodium chloride. The degree of neutralization is preferably from 5 to 60 mol %, preferably from 10 to 40 mol %, more preferably from 20 to 30 mol %, based on the monomers containing the acid groups. This neutralization may be effected before and/or during the polymerization, preferably before the polymerization.

The base is added in such a way that the temperature in the apparatus does not rise above 60° C., and is preferably between 20 and 35° C., and the pH is 4-13. The heat of neutralization is preferably removed by cooling the vessel with the aid of internal cooling coils or via jacket cooling.

The reaction mixture: neutralization liquid ratio is generally 1:0.1-1, preferably 1:0.2-0.8, more preferably 1:0.3-0.7.

With regard to the apparatus, the same applies as was stated above.
h) If a solvent is present in the reaction mixture, it may be substantially removed by distillation. Preference is given to removing any solvent present from the reaction mixture after washing and/or neutralization; however, this may also if desired be done before the wash or neutralization.

To this end, the reaction mixture is admixed with such an amount of storage stabilizer, preferably hydroquinone monomethyl ether, that 100-500, preferably 200-500 and more preferably 200400 ppm thereof are present in the target ester (residue) after removal of the solvent.

The distillative removal of the majority of solvent is effected, for example, in a stirred tank having jacket heating and/or internal heating coils under reduced pressure, for example at 20-700 mbar, preferably 30 to 500 and more preferably 50-150 mbar and a temperature of 40-80° C.

It will be appreciated that the distillation may also be effected in a falling-film or thin-film evaporator. To this end, the reaction mixture, preferably repeatedly in a cycle, is conducted through the apparatus under reduced pressure, for example at 20-700 mbar, preferably from 30 to 500 mbar and more preferably 50-150 mbar and a temperature of 40-80° C.

Advantageously, an inter gas, preferably an oxygenous gas, more preferably air or a mixture of air and nitrogen (lean air) may be introduced into the distillation apparatus, for example 0.1-1, preferably 0.2-0.8 and more preferably 0.3-0.7 m³/m³h, based on the volume of the reaction mixture.

After the distillation, the residual solvent content in the residue is generally below 5% by weight, preferably 0.5-5% and more preferably from 1 to 3% by weight.

The solvent removed is condensed and preferably reused.

If necessary, in addition to or instead of the distillation h), a solvent stripping i) may be carried out.

To this end, the target ester which still contains small amounts of solvent is heated to 50-90° C., preferably 80-90° C., and the remaining amounts of solvent are removed in a suitable apparatus with a suitable gas. This may also be promoted if appropriate by applying a vacuum.

Suitable apparatus are, for example, columns of design known per se which have the customary internals, for example trays, random packings or structured packings, preferably random packings. Useful column internals are in principle all common internals, for example trays, structured packings and/or random packings. Of the trays, preference is given to bubble-cap trays, sieve trays, valve trays, Thormann trays and/or dual-flow trays; of the beds, preference is given to those having rings, spirals, saddles, Raschig, Intos or Pall rings, barrel saddles or Intalox saddles, Top-Pak, etc., or braids.

Also conceivable here is a falling-film, thin-film or wiped-film evaporator, for example a Luwa, Rotafilm or Sambay evaporator, which may be equipped, for example, with a demister as a splashguard.

Suitable gases are gases inert under the stripping conditions, preferably oxygenous gases, more preferably air or mixtures of air and nitrogen (lean air) or steam, especially those which are heated to from 50 to 100° C.

The amount of stripping gas is preferably 5-20, more preferably 10-20 and most preferably from 10 to 15 m³/m³h, based on the volume of the reaction mixture.

If necessary, the ester may be subjected to a filtration j) at any stage in the workup process, preferably after washing/neutralization and any removal of solvent effected, in order to remove precipitated traces of salts and any decolorizing agent present therein.

In one conceivable embodiment, the esterification a) of the polyalcohol A with the carboxylic acid B is carried out in a molar excess, as detailed above, of at least 2.5:1 in the presence of at least one esterification catalyst C and at least one polymerization inhibitor D without a solvent which forms an azeotrope with water.

In a preferred embodiment, the carboxylic acid B used in excess is substantially not removed, i.e. only that portion of carboxylic acid B is removed from the reaction mixture which is determined by the volatility at the temperature employed and, furthermore, no measures are carried out to remove the carboxylic acid, for example distillative, rectificative, extractive, for example washing, absorptive, for example passing over activated carbon or over ion exchangers, and/or chemical steps, for example scavenging of the carboxylic acid with epoxides.

In an alternative embodiment, the carboxylic acid B present in the reaction mixture is removed from the reaction mixture to an extent of not more than 75% by weight, preferably not more than 50% by weight, more preferably not more than 25% by weight, even more preferably not more than 10% by weight and in particular not more than 5% by weight, based on the carboxylic acid B present in the reaction mixture after the end of the reaction.

In a further alternative embodiment, it is possible to dispense with stage b), so that only that proportion of water of reaction and carboxylic acid B is removed from the reaction mixture which is determined by the volatility at the temperature employed. This may preferably be prevented by substantially complete condensation.

In this embodiment, the esterification catalyst C used substantially also remains in the reaction mixture.

In that case, the thus obtained reaction mixture preferably has an acid number to DIN EN 3682 of at least 25 mg KOH/g of reaction mixture, more preferably from 25 to 80 and most preferably from 25 to 50 mg KOH/g.

In this case, preference is given to dispensing with a prewash or postwash e) or g); merely a filtration step j) may be sensible.

Subsequently, the reaction mixture may be diluted in step c); in this case, it is converted to the hydrogel preferably within 6 hours, more preferably within 3 hours. It may preferably be neutralized in step f).

The sequence of steps c), j) and f) is arbitrary.

The reaction mixtures from the preparation of a (meth)acrylic ester of a polyalcohol and especially the purified (meth)acrylic esters which contain at least one 6-chromanol derivative of the formula (III) may find use, for example,
as a free-radical crosslinker of water-absorbent hydrogels,
as the starting material for the preparation of polymer dispersions,
as the starting material for the preparation of polyacrylates (except hydrogels),
as a coatings raw material or
as a cement additive.

They are preferably used as a coatings raw material, for example in radiative curing, and more preferably as a free-radical crosslinker of water-absorbent hydrogels.

It is advantageous that the 6-chromanol derivatives of the formula (III) are nontoxic and thus particularly suitable for use in water-absorbent hydrogels.

The thus obtainable esterification products may be used as free-radical crosslinkers in hydrogels substantially without further purification, particularly without substantial removal of the excess of carboxylic acid B and of the content of esterification catalyst C.

In this document, unless mentioned otherwise, crosslinking refers to free-radical crosslinking (gel crosslinking, internal crosslinking, crosslinking of linear or slightly crosslinked polymer). This crosslinking can be effected via free-radical or cationic polymerization mechanisms, or other esterification or transesterification mechanisms, for example Michael addition, preferably by free-radical polymerization.

Hydrogel-forming polymers which absorb aqueous liquids are preferably those having an absorption of distilled water of at least their own weight, preferably 10 times their own weight; this absorption is preferably also achieved under a pressure of 0.7 psi.

Especially suitable for use as free-radical crosslinkers of water-absorbent hydrogels are especially those reaction mixtures which have a water solubility (at 25° C. in distilled water) of at least 5% by weight, preferably at least 10% by weight, more preferably at least 20% by weight, even more preferably at least 30% by weight and in particular of at least 50% by weight.

The present invention further provides a process for preparing a crosslinked hydrogel, comprising the steps of
a) reacting a polyalcohol A with at least one ethylenically unsaturated carboxylic acid B in the presence of at least one esterification catalyst C and at least one polymerization inhibitor D, and also, if appropriate, a solvent E which forms an azeotrope with water, to form an ester F,
b) if appropriate, removing at least a portion of the water formed in a) from the reaction mixture, b) being effected during and/or after a),
f) if appropriate, neutralizing the reaction mixture,
h) if a solvent E has been used, removing the solvent if appropriate by distillation, and/or
i) stripping with a gas inert under the reaction conditions,
k) polymerizing the reaction mixture from one of stages a) to i), if passed through, with, if appropriate, additional monoethylenically unsaturated compounds N, and also, if appropriate, at least one further copolymerizable hydrophilic monomer M in the presence of at least one free-radical initiator K and, if appropriate, at least one graft base L,
l) if appropriate, postcrosslinking the reaction mixture obtained from k),
m) drying the reaction mixture obtained from k) or l) and
n) if appropriate, grinding and/or sieving the reaction mixture obtained from k), I) or m),
   which comprises using, as the polymerization inhibitor D, at least one 6-chromanol derivative of the formula (III).
k) The reaction mixture from the esterification, including workup steps thereof, if they have been passed through, for example the reaction mixture from f), or, when f) is dispensed with, from b), or, when b) is dispensed with, the reaction mixture from a) may, if appropriate be admixed with additional monoethylenically unsaturated compounds N which do not bear any acid groups, but are copolymerizable with the hydrophilic monomers M, and may then be polymerized to prepare water-absorbent hydrogels in the presence of at least one free-radical initiator K and, if appropriate, at least one graft base L. It may be advantageous to
l) postcrosslink the reaction mixture from k).

Suitable hydrophilic monomers M for preparing k) these hydrophilic, highly swellable hydrogels are, for example, polymerizable acids such as acrylic acid, methacrylic acid, ethacrylic acid, α-chloroacrylic acid, crotonic acid, maleic acid, maleic anhydride, vinylsulfonic acid, vinylphosphonic acid, maleic acid, maleic anhydride, fumaric acid, itaconic acid, citraconic acid, mesaconic acid, glutaconic acid, aconitic acid, allylsulfonic acid, sulfoethyl acrylate, sulfomethacrylate, sulfopropyl acrylate, sulfopropyl methacrylate, 2-hydroxy-3-acryloyloxypropylsulfonic acid, 2-hydroxy-3-methacryloyloxypropylsulfonic acid, allylphosphonic acid, styrenesulfonic acid, 2-acrylamido-2-methylpropanesulfonic acid, 2-acrylamido-2-methylpropanephosphonic acid and also their amides, hydroxyalkyl esters, and esters and amides containing amine or ammonium groups. These monomers can be used alone or in a mixture with one other. In addition, water-soluble N-vinylamides or else diallyldimethyl-ammonium chloride.

Preferred hydrophilic monomers are compounds of the formula (VIII) 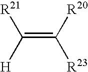
where
R²⁰ is hydrogen, methyl or ethyl,
R²¹ is —COOR²⁴, a sulfonyl group or phosphonyl group, a phosphonyl group esterified with a (C₁-C₄)-alkanol, or a group of the formula (IX) 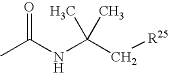
R²³ is hydrogen, methyl, ethyl or a carboxyl group,
R²⁴ is hydrogen, C₁-C₄-alkyl or hydroxy-(C₁-C₄)-alkyl and
R²⁵ is a sulfonyl group, a phosphonyl group or a carboxyl group.

Examples of (C₁-C₄)-alkanols are methanol, ethanol, n-propanol and n-butanol.

Particularly preferred hydrophilic monomers are acrylic acid and methacrylic acid.

To optimize properties, it can be sensible to use additional monoethylenically unsaturated compounds N which do not bear an acid group but are copolymerizable with the monomers bearing acid groups. Such compounds include, for example, the amides and nitriles of monoethylenically unsaturated carboxylic acids, for example acrylamide, methacrylamide and N-vinylformamide, N-vinylacetamide, N-methylvinyl-acetamide, acrylonitrile and methacrylonitrile. Examples of further suitable compounds are vinyl esters of saturated C₁- to C₄-carboxylic acids such as vinyl formate, vinyl acetate or vinyl propionate, alkyl vinyl ethers having at least 2 carbon atoms in the alkyl group, for example ethyl vinyl ether or butyl vinyl ether, esters of monoethylenically unsaturated C₃- to C₆-carboxylic acids, for example esters of monohydric C₁- to C₁₋₈-alcohols and acrylic acid, methacrylic acid or maleic acid, monoesters of maleic acid, for example monomethyl maleate, N-vinyllactams such as N-vinylpyrrolidone or N-vinylcaprolactam, acrylic and methacrylic esters of alkoxylated monohydric saturated alcohols, for example of alcohols having from 10 to 25 carbon atoms which have been reacted with from 2 to 200 mol of ethylene oxide and/or propylene oxide per mole of alcohol, and also monoacrylic esters and monomethacrylic esters of polyethylene glycol or polypropylene glycol, the molar masses (Mₙ) of the polyalkylene glycols being up to 2000, for example. Further suitable monomers are styrene and alkyl-substituted styrenes such as ethylstyrene or tert-butylstyrene.

These monomers bearing no acid groups may also be used in mixture with other monomers, for example mixtures of vinyl acetate and 2-hydroxyethyl acrylate in any ratio. These monomers bearing no acid groups are added to the reaction mixture in amounts within the range from 0 to 50% by weight, preferably less than 20% by weight.

The crosslinked (co)polymers preferably consist of monoethylenically unsaturated monomers which bear acid groups and have optionally been converted, before or after polymerization, to their alkali metal or ammonium salts, and of 040% by weight, based on their total weight, of monoethylenically unsaturated monomers bearing no acid groups.

The preparation of (meth)acrylic acid (co)polymers, polyacrylic acids and superabsorbents has been extensively described before and therefore is well known; see, for example, “Modern Superabsorbent Polymer Technology”, F. L. Buchholz and A. T. Graham, Wiley-VCH, 1998.

Preference is given to those hydrogels which are obtained by crosslinking polymerization or copolymerization of monoethylenically unsaturated monomers M bearing acid groups or salts thereof.

In the process for postcrosslinking, the starting polymer is treated with a postcrosslinker and preferably, during or after the treatment, postcrosslinked and dried by raising the temperature, the crosslinker preferably being present in an inert solvent. Inert solvents refer to those which substantially do not react either with the starting polymer or with the postcrosslinker. Preference is given to those solvents which do not react chemically with the starting polymer or with the postcrosslinker to an extent of more than 90%, preferably more than 95%, more preferably more than 99% and especially more than 99.5%.

For postcrosslinking l) and drying m), preference is given to the temperature range between 30 and 250° C., especially 50-200° C.; very particular preference is given to the range between 100-180° C. The surface postcrosslinking solution is preferably applied by spraying onto the polymer in suitable spray mixers. After spraying, the polymer powder is thermally dried, and the crosslinking reaction can take place not only before but also during the drying operation. Preference is given to spraying a solution of the crosslinker in reaction mixers or mixing and drying units, for example Lödige mixers, BEPEX mixers, NAUTA mixers, SHUGGI mixers or PROCESSALL. It is moreover also possible to use fluidized bed dryers.

The drying operation can take place in the mixer itself, by heating of the shell or by blowing in hot air. Equally suitable is a downstream dryer, for example a tray dryer, a rotary tube oven or a heatable screw. However, it is also possible to utilize an azeotropic distillation as the drying method, for example. The preferred residence time at this temperature in the reaction mixer or dryer is below 60 min, more preferably below 30 min.

Preference is given to the above processes in which the starting polymer is a polymeric acrylic acid or a polyacrylate, especially a polymeric acrylic acid or a polyacrylate which has been obtained by free-radical polymerization and for which a polyfunctional ethylenically unsaturated free-radical crosslinker has been used.

Preference is given to those processes in which the substance mixture comprising free radical crosslinkers, i.e. the ester F, and diluents G in a ratio of 0.1-20% by weight, especially 0.5-10% by weight, based on the mass of the starting polymer is used.

Preference is given to those processes in which the free radical crosslinker is used in a dose of 0.01-5.0% by weight, preferably 0.02-3.0% by weight, more preferably 0.03-2.5% by weight, in particular 0.05-1.0% and especially from 0.1% to 0.75% by weight, based on the starting polymer.

The present invention also provides polymers prepared by one of the processes mentioned above and their use in hygiene articles, packaging materials and in nonwovens, and also the use of an abovementioned substance mixture for preparing crosslinked or thermally crosslinkable polymers, especially in paints and varnishes.

The hydrophilic, highly swellable hydrogels to be used (starting polymers) are in particular polymers of (co)polymerized hydrophilic monomers M, graft (co)polymers of one or more hydrophilic monomers M on a suitable grafting base L, crosslinked cellulose or starch ethers or natural products capable of swelling in aqueous fluids, for example guar derivatives. These hydrogels are known to those skilled in the art and are described, for example, in U.S. Pat. No. 4,286,082, DE-C-27 06 135, U.S. Pat. No. 4,340,706, DE-C-37 13 601, DE-C-28 40 010, DE-A-43 44 548, DE-A-40 20 780, DE-A-40 15 085, DE-A-39 17 846, DE-A-38 07 289, DE-A-35 33 337, DE-A-35 03 458, DE-A-42 44 548, DE-A-42 19 607, DE-A-40 21 847, DE-A-38 31 261, DE-A-35 11 086, DE-A-31 18 172, DE-A-30 28 043, DE-A-44 18 881, EP-A-0 801 483, EP-A-0 455 985, EP-A-0 467 073, EP-A-0 312 952, EP-A-0 205 874, EP-A-0 499 774, DE-A 26 12 846, DE-A-40 20 780, EP-A-0 205 674, U.S. Pat. No. 5,145,906, EP-A-0 530 438, EP-A-0 670 073, U.S. Pat. No. 4,057,521, U.S. Pat. No. 4,062,817, U.S. Pat. No. 4,525,527, U.S. Pat. No. 4,295,987, U.S. Pat. No. 5,011,892, U.S. Pat. No. 4,076,663 or U.S. Pat. No. 4,931,497. Also particularly suitable are highly swellable hydrogels from a production process as described in WO 01/38402, and also inorganic/organic hybrid, highly swellable hydrogels as described in DE 198 54 575. The content of the aforementioned patent documents, especially the hydrogels produced by the processes, is explicitly incorporated herein by reference.

Suitable grafting bases L for hydrophilic hydrogels which are obtainable by graft copolymerization of olefinically unsaturated acids can be of natural or synthetic origin. Examples are starch, cellulose or cellulose derivatives and also other polysaccharides and oligosaccharides, polyalkylene oxides, especially polyethylene oxides and polypropylene oxides, and also hydrophilic polyesters.

The water-absorbent polymer can be obtained by free-radical graft copolymerization of acrylic acid or acrylate onto a water-soluble polymer matrix. Nonlimiting examples of suitable water-soluble polymer matrices are alginates, polyvinyl alcohol and polysaccharides, for instance starch. In this type of graft copolymerization, a polyfunctional ethylenically unsaturated free radical crosslinker is used.

The water-absorbent polymer can be an organic/inorganic hybrid polymer formed from a polymeric acrylic acid or polyacrylate on the one hand and a silicate, aluminate or aluminosilicate on the other. More particularly, polymeric acrylic acid or polyacrylate may be used which has been obtained by free-radical polymerization, and for which a polyfunctional ethylenically unsaturated free radical crosslinker has been used, and in whose preparation process a water-soluble silicate or soluble aluminate or mixtures thereof has been used.

Preferred hydrogels are in particular polyacrylates, polymethacrylates and also the graft polymers described in U.S. Pat. No. 4,931,497, U.S. Pat. No. 5,011,892 and U.S. Pat. No. 5,041,496. Very particularly preferred hydrogels are the kneader polymers described in WO 01/38402 and the organic/inorganic hybrid hydrogels based on polyacrylates and described in DE 198 545 75.

The substances which are prepared in accordance with the present invention and can be used as free radical crosslinkers in hydrogels can be used alone or in combination with other crosslinkers, for example internal or surface crosslinkers, for example the following:

Suitable crosslinkers are in particular methylenebisacrylamide, methylenebismethacrylamide, esters of unsaturated mono- or polycarboxylic acids with polyols, such as diacrylate or triacrylate, for example butanediol diacrylate, butanediol dimethacrylate, ethylene glycol diacrylate, ethylene glycol dimethacrylate, and also trimethylolpropane triacrylate, and allyl compounds such as allyl(meth)acrylate, triallyl cyanurate, diallyl maleate, polyallyl esters, tetraallyloxyethane, triallylamine, tetraallylethylenediamine, allyl esters of phosphoric acid and also vinylphosphonic acid derivatives, as described, for example, in EP-A-0 343 427. However, particular preference for use in the process of the present invention is given to hydrogels which are prepared using polyallyl ethers as crosslinkers and by acidic homopolymerization of acrylic acid. Suitable crosslinkers are pentaerythritol triallyl ether, pentaerythritol tetraallyl ether, polyethylene glycol diallyl ether, monoethylene glycol diallyl ether, glycerol diallyl ether, glycerol triallyl ether, polyallyl ethers based on sorbitol and also ethoxylated variants thereof. Particularly preferred crosslinkers further include polyethylene glycol diacrylates, ethoxylated derivatives of trimethylolpropane triacrylate, for example Sartomer SR 9035, and also ethoxylated derivatives of glycerol diacrylate and glycerol triacrylate. It is obviously also possible to use mixtures of the above crosslinkers.

Very particular preference is given to hydrogels which are prepared using an ester F prepared in accordance with the invention as a free radical crosslinker.

The water-absorbent polymer is preferably a polymeric acrylic acid or a polyacrylate. This water-absorbent polymer can be prepared by a process known from the literature. Preference is given to polymers which contain crosslinking comonomers (0.001-10 mol %), but very particular preference is given to polymers which have been obtained by free-radical polymerization and for which a polyfunctional ethylenically unsaturated free radical crosslinker has been used.

The hydrophilic, highly swellable hydrogels can be prepared by polymerization processes known per se. Preference is given to the polymerization in aqueous solution by the gel polymerization method. It involves, as stated above, polymerizing dilute, preferably aqueous and more preferably 15-50% by weight aqueous, solutions of one or more hydrophilic monomers and optionally of a suitable grafting base L in the presence of a free-radical initiator, preferably without mechanical mixing, by utilizing the Trommsdorff-Norrish effect (Makromol. Chem. 1, 169 (1947)). The polymerization reaction may be carried out within the temperature range from 0° C. to 150° C., preferably between 10° C. and 100° C., either at atmospheric pressure or at elevated or reduced pressure. As usual, the polymerization can also be performed in a protective gas atmosphere, preferably under nitrogen. The polymerization may be induced using high-energy electromagnetic rays or the customary chemical polymerization initiators K, for example organic peroxides, such as benzoyl peroxide, tert-butyl hydroperoxide, methyl ethyl ketone peroxide, cumene hydroperoxide, azo compounds such as azobisiso-butyronitrile and also inorganic peroxy compounds such as (NH₄)₂S₂O₈, K₂S₂O₈ or H₂O₂.

They can, if appropriate, be used in combination with reducing agents, for example ascorbic acid, sodium hydrogensulfite and iron(II) sulfate or redox systems where the reducing component present is an aliphatic and aromatic sulfinic acid, such as benzenesulfonic acid and toluenesulfonic acid or derivatives of these acids, for example Mannich adducts of sulfinic acids, aldehydes and amino compounds, as described in DE-C-1 301 566. The performance properties of the polymers can be further improved by postheating the polymer gels in the temperature range from 50° to 130° C., preferably from 70° to 100° C., for several hours.

The resulting gels are neutralized to the extent of 0-100 mol %, preferably 25-100 mol % and more preferably 50-85 mol %, based on monomer used, for which the customary neutralizing agents can be used, preferably alkali metal hydroxides, alkali metal oxides or the corresponding alkali metal carbonates, but more preferably sodium hydroxide, sodium carbonate and sodium hydrogencarbonate.

Neutralization is typically achieved by mixing in the neutralizing agent as an aqueous solution or else preferably as a solid. For this purpose, the gel is mechanically comminuted, for example by means of a meat grinder, and the neutralizing agent is sprayed on, scattered on or poured on and then carefully mixed in. The gel mass obtained can then be repeatedly passed through the meat grinder for homogenization. The neutralized gel mass is then dried with a belt dryer or roll dryer until the residual moisture content is preferably below 10% by weight, especially below 5% by weight.

The polymerization as such can also be carried out by any other process described in the literature. More particularly, the neutralization of the acrylic acid can also be carried out before the polymerization, as described above in step f). The polymerization can then be carried out in a belt reactor known to those skilled in the art or a kneading reactor, continuously or else batchwise. When the polymerization is carried out in a belt reactor, initiation by means of electromagnetic radiation, preferably by means of UV radiation, or alternatively initiation using a redox initiator system is particularly preferred. Very particular preference is also given to a combination of the two methods of initiation: electromagnetic radiation and chemical redox initiator system simultaneously.
n) Afterward, the dried hydrogel can be ground and sieved, in which case it is customary to use roll mills, pin mills or sieving mills for the grinding. The preferred particle size of the sieved hydrogel is preferably in the 45-1000 μm range, more preferably at 45-850 μm, even more preferably at 200-850 μm, and most preferably at 300-850 μm. These ranges preferably cover 80% by weight of the particles, especially 90% by weight of the particles. The size distribution can be determined using established laser methods.

It will be appreciated that chromanols may also be used for stabilization in processes for preparing (meth)acrylic esters of monoalcohols in an esterification.

Preferred monoalcohols are monoalcohols having from 1 to 8 carbon atoms, preferably those having from 1 to 4 and more preferably those having from 1 to 2 carbon atoms. Particularly preferred monoalcohols are methanol, ethanol, n-propanol, isopropanol, n-butanol, isobutanol and 2-ethylhexanol; very particular preference is given to methanol, ethanol and n-butanol which lead to correspondingly lower (meth)acrylic esters.

Examples of high-boiling monoalcohols are tert-butylcyclohexanol, lauryl alcohol (1-dodecanol), myristyl alcohol (1-tetradecanol), cetyl alcohol (1-hexadecanol), stearyl alcohol (1-octadecanol), 9-cis-octadecen-1-ol (oleyl alcohol), 9-trans-octadecen-1-ol (erucyl alcohol), 9-cis-octadecene-1,12-diol (ricinol alcohol), all-cis-9,12-octadecadien-1-ol (linoleyl alcohol), all-cis-9,12,15-octadecatrien-1-ol (linolenyl alcohol), 1-eicosanol (arachidyl alcohol), 9-cis-eicosen-1-ol (gadoleyl alcohol), 1-docosanol (behenyl alcohol), 1,3-cis-docosen-1-ol (erucyl alcohol) and 1,3-trans-docosen-1-ol (brassidyl alcohol).

The alcohols may also be ethoxylated and/or propoxylated alcohols, and also mixed ethoxylated/propoxylated alcohols such as
R²⁶—(O—CH₂—CH₂)ₓ—OH or
R²⁶—(O—CH(CH₃)—CH₂)ₓ—OH or R—(O—CH₂—CH(CH₃))ₓ—OH,
where
R²⁶ is C₁-C₂₂-alkyl and
x is an integer between 1 and 20.

Examples of R²⁶ are methyl, ethyl, isopropyl, n-propyl, allyl, n-butyl, isobutyl, sec-butyl, tert-butyl, n-hexyl, n-heptyl, n-octyl, n-decyl, n-dodecyl, n-tetradecyl, n-hexadecyl, n-octadecyl or n-eicosyl.

Such (meth)acrylic esters of monoalcohols are frequently prepared continuously. Processes for preparing these (meth)acrylic esters are described, for example, in EP-A1 733 617, DE-B 25 48 561, DE-A1 196 04 252, DE-A1 196 04 253, DE-A1 196 04 267 and WO 00/78702, which are incorporated in the scope of this disclosure by reference.

ppm data and percentages used in this document, unless stated otherwise, relate to percentages by weight and ppm by weight.

The examples which follow are intended to illustrate the invention, but not restrict it to these examples.

### EXAMPLES

### Example 1

45 parts of ethoxylated trimethylolpropane having molecular weight Mn of approx. 450, 55 parts of acrylic acid, 70 parts of methylcyclohexane, 1 part of hydroquinone monomethyl ether, 0.4 part of 2,2,5,7,8-pentamethyl-6-chromanol and 1 part of sulfuric acid were combined, heated to reflux under a nitrogen-oxygen mixture having 6% by volume of oxygen and water is removed until no more distills over. Afterward, the azeotroping agent was distilled off. The degree of esterification determined by IR was approx. 80%.

The deposits of polymer on the tank were determined and the HAZEN color number (DIN ISO 6271) was measured.

Deposits: 0 parts color number: 71

### Example 2

45 parts of ethoxylated trimethylolpropane having molecular weight Mn of approx. 450, 55 parts of acrylic acid, 70 parts of methylcyclohexane, 1 part of hydroquinone monomethyl ether, 0.2 part of 2,2,5,7,8-pentamethyl-6-chromanol and 1 part of sulfuric acid were combined, heated to reflux under a nitrogen-oxygen mixture having 6% by volume of oxygen and water is removed until no more distills over. Afterward, the azeotroping agent was distilled off. The degree of esterification determined by IR was approx. 80%.

Deposits: 1 part color number 70

### Example 3

97 parts of polytetrahydrofuran of Mn of approx. 650, 55 parts of acrylic acid, 70 parts of methylcyclohexane, 1 part of hydroquinone monomethyl ether, 0.4 part of 2,2,5,7,8-pentamethyl-6-chromanol and 1 part of sulfuric acid were combined, heated to reflux under a nitrogen-oxygen mixture having 6% by volume of oxygen and water is removed until no more distills over. Afterward, the azeotroping agent was distilled off. The degree of esterification determined by IR was approx. 75%.

Deposits: 0 color number: 85

### Comparative Example 1

45 parts of ethoxylated trimethylolpropane having molecular weight Mn of approx. 450, 55 parts of acrylic acid, 70 parts of methylcyclohexane, 1 part of hydroquinone monomethyl ether, 0.4 part of Kerobit TBK (2,6-di-t-butylcresol) and 1 part of sulfuric acid were combined, heated to reflux under a nitrogen-oxygen mixture having 6% by volume of oxygen and water is removed until no more distills over. Afterward, the azeotroping agent was distilled off. The degree of esterification determined by IR was approx. 80%.

Deposits: 2 parts color number: 131

### Comparative Example 2

45 parts of ethoxylated trimethylolpropane having molecular weight Mn of approx. 450, 55 parts of acrylic acid, 70 parts of methylcyclohexane, 1 part of hydroquinone monomethyl ether, 0.4 part of vitamin E and 1 part of sulfuric acid were combined, heated to reflux under a nitrogen-oxygen mixture having 6% by volume of oxygen and water is removed until no more distills over. Afterward, the azeotroping agent was distilled off. The degree of esterification determined by IR was approx. 80%.

Deposits: 3 parts color number:51

### Comparative Example 3

45 parts of ethoxylated trimethylolpropane having molecular weight Mn of approx. 450, 55 parts of acrylic acid, 70 parts of methylcyclohexane, 1 part of hydroquinone monomethyl ether, 0.4 part of Irganox® XP 621 (Ciba Spezialitätenchemie, commercially available stabilizer mixture of antioxidant and peroxide decomposer) and 1 part of sulfuric acid were combined, heated to reflux under a nitrogen-oxygen mixture having 6% by volume of oxygen and water is removed until no more distills over. Afterward, the azeotroping agent was distilled off. The degree of esterification determined by IR was approx. 80%.

Deposits: 17 parts color number 88

### Comparative Example 4

45 parts of ethoxylated trimethylolpropane having molecular weight Mn of approx. 450, 55 parts of acrylic acid, 70 parts of methylcyclohexane, 1 part of hydroquinone monomethyl ether, 0.4 part of trimethylhydroquinone and 1 part of sulfuric acid were combined, heated to reflux under a nitrogen-oxygen mixture having 6% by volume of oxygen and water is removed until no more distills over. Afterward, the azeotroping agent was distilled off. The degree of esterification determined by IR was approx. 80%.

Deposits: 0 color number: 887

### Comparative Example 5

45 parts of ethoxylated trimethylolpropane having molecular weight Mn of approx. 450, 55 parts of acrylic acid, 70 parts of methylcyclohexane, 1 part of hydroquinone monomethyl ether, 0.4 part of 2-t-butylhydroxy-4-anisole and 1 part of sulfuric acid were combined, heated to reflux under a nitrogen-oxygen mixture having 6% by volume of oxygen and water is removed until no more distills over. Afterward, the azeotroping agent was distilled off. The degree of esterification determined by IR was approx. 80%.

Deposits: 1 part color number: 148

1. A process for preparing an ester F of a polyalcohol A with at least one α,β-ethylenically unsaturated carboxylic acid B, comprising the steps of
a) reacting a polyalcohol A with at least one α,β-ethylenically unsaturated carboxylic acid B in the presence of at least one esterification catalyst C and at least one polymerization inhibitor D, and a solvent E which forms an azeotrope with water, to form an ester F,
b) removing at least a portion of the water formed in a) from the reaction mixture, b) during and after a) or after step a),
f) neutralizing the reaction mixture,
h) removing the solvent by distillation, and
i) stripping with a gas inert under the reaction conditions or both steps h) and i), which comprises using, as the polymerization inhibitor D, at least one 6-chromanol derivative of the formula (III) 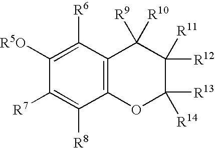
where
R⁵, R⁶, R⁷, R⁸, R⁹, R¹⁰, R¹¹, R¹², R¹³ and R¹⁴ are each independently hydrogen, C₁-C₄-alkyl, and
R⁵ is additionally C₁-C₄-alkylcarbonyl, C₁-C₄-alkyloxycarbonyl, C₆-C₁₂-arylcarbonyl or
C₆-C₁₂-aryloxycarbonyl,
and R¹³ is additionally chlorine.

2. A process for preparing a crosslinked hydrogel, comprising the steps of
q) reacting a polyalcohol A with at least one α,β-ethylenically unsaturated carboxylic acid B in the presence of at least one esterification catalyst C and at least one polymerization inhibitor D, and a solvent E which forms an azeotrope with water, to form an ester F,
b) removing at least a portion of the water formed in a) from the reaction mixture, b) during and after a) or after step a),
f) neutralizing the reaction mixture,
h) removing the solvent by distillation, and
i) stripping with a gas inert under the reaction conditions or both steps h) and i),
k) polymerizing the reaction mixture from one of stages a) to i), with, additional monoethylenically unsaturated compounds N, and also, at least one further copolymerizable hydrophilic monomer M in the presence of at least one free-radical initiator K and, at least one graft base L,
l) postcrosslinking the reaction mixture obtained from k),
m) drying the reaction mixture obtained from k) or l) and
n) grinding and sieving the reaction mixture obtained from k), l) or m) or grinding or sieving said reaction mixture,
which comprises using, as the polymerization inhibitor D, at least one 6-chromanol derivative of the formula (III).

3. The process according to claim 1, wherein R⁵ and R⁹ to R¹² in formula (III) are each hydrogen, R⁶, R⁷ and R⁸ are each independently hydrogen or methyl, and R¹³ and R¹⁴ are each methyl.

4. The process according to claim 1, wherein at least one 6-chromanol derivative is selected from the group consisting of 2,2,5,7,8-pentamethyl-6-chromanol, 2,2,5,7-tetramethyl-6-chromanol, 2,2,5,8-tetramethyl-6-chromanol, 2,2,7,8-tetramethyl-6-chromanol, 2,2,5-trimethyl-6-chromanol, 2,2,7-trimethyl-6-chromanol and 2,2,8-trimethyl-6-chromanol.

5. The process according to claim 1, wherein at least one of reaction steps a) and b) is carried out in the presence of an oxygenous gas.

6. The process according to any of the preceding claims claim 1, wherein the polyalcohol A is selected from the group consisting of trimethylolbutane, trimethylolpropane, trimethylolethane, neopentyl glycol, neopentyl hydroxypivalate, pentaerythritol, glycerol, 1,2-ethylene glycol, 1,2-propylene glycol, 2-ethyl-1,3-propanediol, 2-methyl-1,3-propanediol, hydroquinone, bisphenol A, bisphenol F, bisphenol B, 2,2-bis(4-hydroxycyclohexyl)propane, 1,1-, 1,2-, 1,3- and 1,4-cyclohexanedimethanol, 1,2-, 1,3- or 1,4-cyclohexanediol, but-2-ene-1,4-diol and but-2-yne-1,4-diol, each of which may optionally be alkoxylated.

7. The process according to claim 1, wherein, a C₁-C₄-alkyl ester of a carboxylic acid B is used and, a transesterification catalyst.

8. A crosslinked hydrogel prepared by the process according to claim 2.

9. A crosslinked hydrogel comprising at least one hydrophilic monomer M in copolymerized form, crosslinked with a reaction mixture comprising an ester F, prepared by the process according to claim 1.

10. The crosslinked hydrogel according to claim 8, comprising at least one 6-chromanol derivative of the formula (III).

11. A method of using the crosslinked hydrogel according to claim 8 in hygiene articles, packaging materials and in nonwovens.

12. A method of using the reaction mixtures from the preparation of a (meth)acrylic ester of a polyalcohol or of a purified (meth)acrylic ester according to claim 1, each of which comprise at least one 6-chromanol derivative of the formula (III) as defined in claim 1, as free-radical crosslinkers of water-absorbent hydrogels.

13. A method of using the 6-chromanol derivatives of the formula (III) as defined in claim 1 as the stabilizer in the preparation of (meth)acrylic esters.

14. The method according to claim 12, wherein the (meth)acrylic esters are used as free-radical crosslinkers in hydrogels.

15. A substance mixture comprising
at least one 6-chromanol derivative of the formula (III) as defined in claim 1 and
at least one stabilizer selected from the group consisting of phenothiazine, hydroquinone, hydroquinone monomethyl ether and hypophosphorous acid.
